(19) United States
(12) Patent Application Publication
(10) Pub. No.: US 2007/0239278 A1
(43) Pub. Date: Oct. 11, 2007

(54) Intervertebral prosthetic devices and methods

(75) Inventor: Eric Steven Heinz, Memphis, TN (US)

Correspondence Address: HESLIN ROTHENBERG FARLEY & MESITI P.C., 5 COLUMBIA CIRCLE, ALBANY, NY, 12203, US

(73) Assignee: SDGI Holdings, Inc., Wilmington, DE (US)

(21) Appl. No.: 11/398,874

(22) Filed: Apr. 6, 2006

(51) Int. Cl. A61F 2/44; A61B 17/84

(52) U.S. Cl. 623/17.15; 606/75

(57) ABSTRACT

Intervertebral prosthetic devices and methods are provided which include an arthroplasty implant configured to reside within an intervertebral space between first and second vertebral bodies, and at least one implant-to-vertebra staple. Each staple, which includes first and second prongs, is formed of a shape memory material having a memorized state and a deformed state. The arthroplasty implant includes at least one vertebral support plate configured to engage at least one of the first and second vertebral bodies, and configured to receive the first prong of the staple(s). When the arthroplasty implant is disposed in the intervertebral space, the staple(s) couples the at least one vertebral support plate to the at least one first and second vertebral bodies when in the deformed state, and thereafter transitions to the memorized state resulting in compressive force being applied between the vertebral support plate(s) and the at least one first and second vertebral bodies.

### CROSS-REFERENCE TO RELATED APPLICATIONS/PATENTS

This application contains subject matter which is related to the subject matter of the following applications/patents, which are hereby incorporated herein by reference in their entirety:

“Intervertebral Spinal Implant Devices and Methods of Use”, Heinz et al., U.S. Ser. No.: 11/343,954 filed Jan. 31, 2006;

“Intervertebral Prosthetic Disc”, Heinz et al., U.S. Ser. No.: 11/343,935, filed Jan. 31, 2006;

“Constrained Artificial Spinal Disc”, Marik et al., U.S. Ser. No.: 10/806,961, filed Mar. 23, 2004, and published on Sep. 29, 2005 as Patent Application Publication No. US 2005/0216086 A1;

“Intervertebral Prosthetic Joint”, Eisermann et al., U.S. Ser. No.: 10/620,529, filed Jul. 16, 2003, and published on Apr. 15, 2004 as Patent Application Publication No. US 2004/0073312 A1;

“Method for the Correction of Spinal Deformities Through Vertebral Body Tethering Without Fusion”, Ogilvie et al., U.S. Ser. No.: 09/905,018, filed Jul. 13, 2001, and published on Jan. 17, 2002 as Patent Application Publication No. US 2002/0007184 A1;

“Shape Memory Alloy Staple”, Ogilvie et al., U.S. Pat. No. 6,773,437 B2, issued Aug. 10, 2004;

“Intervertebral Prosthetic Joint”, Eisermann et al., U.S. Pat. No. 6,740,118 B2, issued May 25, 2004;

“Method for the Correction of Spinal Deformities Through Vertebral Body Tethering Without Fusion”, Ogilvie et al., U.S. Pat. No. 6,616,669 B2, issued Sep. 9, 2003; and

“Shape Memory Alloy Staple”, Ogilvie et al., U.S. Pat. No. 6,325,805 B1, issued Dec. 4, 2001.

### TECHNICAL FIELD

The present invention relates generally to spinal implants and methods, and more particularly, to intervertebral prosthetic joint devices and methods for use in total or partial replacement of a natural intervertebral disc.

### BACKGROUND OF THE INVENTION

In the treatment of disease, injuries and malformations affecting spinal motion segments, and especially those affecting disc tissue, it has been known to remove some or all of a degenerated, ruptured or otherwise failing disc. In cases involving intervertebral disc tissue that has been removed, or is otherwise absent from a spinal motion segment, corrective measures are typically desirable.

In one approach, the two adjacent vertebrae are fused together using transplanted bone tissue, an artificial fusion component, or other compositions or devices. Spinal fusion procedures, however, have raised concerns in the medical community that the biomechanical rigidity of the intervertebral fusion may predispose neighboring spinal motion segments to rapid deterioration. Unlike a natural intervertebral disc, spinal fusion prevents the fused vertebrae from pivoting and rotating with respect to one another. Such lack of mobility tends to increase stress on adjacent spinal motion segments. Additionally, conditions may develop within adjacent spinal motion segments, including disc degeneration, disc herniation, instability, spinal stenosis, spondylosis and facet joint arthritis as a result of the spinal fusion. Consequently, many patients may require additional disc removal and/or another type of surgical procedure as a result of the spinal fusion. Alternatives to spinal fusion are therefore desirable.

Several different types of intervertebral disc arthroplasty devices have been proposed for preventing the collapse of the intervertebral space between adjacent vertebrae while maintaining a certain degree of stability and range of pivotal and rotational motion therebetween. Such devices typically include two or more articular elements that couple to respective upper and lower vertebrae. The articular elements are conventionally anchored to the upper and lower vertebrae by a number of methods, including the use of bone screws that pass through corresponding openings in each of the elements and thread into vertebral bone, and/or by the inclusion of spikes or teeth that penetrate the vertebral endplates to inhibit migration or expulsion of the device. The articular elements are typically configured to allow the elements, and correspondingly the adjacent vertebrae, to pivot and/or rotate relative to one another.

Existing intervertebral disc arthroplasty devices are relatively difficult to implant between adjacent vertebrae. To implant such devices, the adjacent vertebrae are spread apart a distance that is somewhat greater than the normal distance separating the vertebrae so that the device can be maneuvered between the vertebrae and the anchors can be engaged to the vertebral endplates. Such an operation presents a risk of injury to the vertebrae caused by misplacement and/or scratching of the vertebral endplates or other tissue by the anchors. The operation also presents a risk of injury resulting from over-distraction of the intervertebral space. Further, existing arthroplasty devices requiring the threading of bone screws into the adjacent vertebrae require precise placement and orientation of the bone screws to provide adequate anchoring and to avoid injury to adjacent tissue or vertebral structures.

Another disadvantage of existing arthroplasty devices is that they typically may not be manipulated in position after being inserted into the disc space. Most arthroplasty devices employ teeth, keels or spikes which prevent manipulation of the implant once positioned within the intervertebral space.

Thus, there remains a need in the art for improved intervertebral prosthetic disc devices and methods of use thereof. The devices and methods disclosed herein address these needs.

### SUMMARY OF THE INVENTION

The shortcomings of the prior art are overcome and additional advantages are provided, in one aspect, through provision of an intervertebral prosthetic device which includes at least one staple comprising at least two prongs, including a first prong and a second prong, and at least one component configured to reside within an intervertebral space between a first vertebral body and a second vertebral body. The at least one component includes at least one vertebral bearing surface configured to engage at least one of the first vertebral body and the second vertebral body, and to facilitate repositioning of the at least one component within the intervertebral space prior to engageable coupling thereof to the at least one of the first vertebral body and the second vertebral body employing the at least one staple. In addition, the at least one component is configured to receive the first prong of the at least one staple. When the at least one component is disposed in the intervertebral space, the at least one staple couples the at least one vertebral bearing surface of the at least one component to the at least one first vertebral body and second vertebral body, with the first prong thereof being received by the at least one component and the second prong engaging the at least one first vertebral body and second vertebral body.

In another aspect, an intervertebral prosthetic device is provided which includes at least one staple with at least two prongs, including a first prong and a second prong, and an arthroplasty implant configured to reside within an intervertebral space between a first vertebral body and a second vertebral body. The at least one staple is formed of a shape memory material having a memorized state and a deformed state. The arthroplasty implant includes at least one vertebral support plate configured to engage at least one of the first vertebral body and the second vertebral body. The at least one vertebral support plate is further configured to receive the first prong of the at least one staple. When the arthroplasty implant is disposed in the intervertebral space, the at least one staple couples the at least one vertebral support plate of the arthroplasty implant to the at least one first vertebral body and second vertebral body when in the deformed state, and thereafter transitions to the memorized state resulting in compressive force being applied between the at least one vertebral support plate and the at least one first vertebral body and second vertebral body.

In a further aspect, a method of providing a prosthetic intervertebral disc replacement is disclosed. The method includes obtaining an intervertebral prosthetic device comprising at least one staple and an arthroplasty implant, the at least one staple including a first prong and a second prong, and being formed of a shape memory material having a memorized state and a deformed state, and the arthroplasty implant being configured to reside within an intervertebral space between a first vertebral body and a second vertebral body, the arthroplasty implant including at least one vertebral support plate configured for engaging at least one of the first vertebral body and the second vertebral body, and the at least one vertebral support plate being configured to receive the first prong of the at least one staple. The method further includes: preparing the intervertebral disc space to receive the intervertebral prosthetic device; positioning the arthroplasty implant within the intervertebral space; inserting the at least one staple in the deformed state into engageable contact with the at least one vertebral support plate and the at least one first vertebral body and second vertebral body; and allowing the at least one staple to at least partially return to the memorized state, thereby applying a compressive force between the at least one vertebral support plate and the at least one first vertebral body and second vertebral body.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a lateral view of a portion of a spine, including two vertebral bodies and a disc disposed therebetween;

FIG. 1A is a side elevational view of one embodiment of a spinal arthroplasty implant;

FIG. 2 is an elevational view of an alternate embodiment of a spinal arthroplasty implant, in accordance with an aspect of the present invention;

FIG. 2A is a cross-sectional lateral view of the arthroplasty implant of FIG. 2 taken along line 2A-2A;

FIG. 3 is a lateral view of a spine portion and the arthroplasty implant of FIG. 2A (shown in cross-sectional view) disposed in an intervertebral space between two vertebral bodies, in accordance with an aspect of the present invention;

FIG. 4 depicts one embodiment of an implant-to-vertebra staple, formed of shape memory material, for use with an arthroplasty implant such as depicted in FIGS. 2 & 3, with the deformed insertion position of the staple prongs shown in phantom, in accordance with an aspect of the present invention;

FIG. 5 is a lateral view of a spine portion and partially exploded view of one embodiment of an intervertebral prosthetic device, including the arthroplasty implant of FIGS. 2 & 3, and two implant-to-vertebra staples in deformed position, in accordance with an aspect of the present invention;

FIG. 6 is a lateral view of the spine portion of FIG. 5 showing the intervertebral prosthetic device with the staples thereof inserted in their deformed position into the arthroplasty implant and the respective vertebral bodies, in accordance with an aspect of the present invention;

FIG. 7 is a lateral view of the spine portion of FIG. 6, after the staple prongs have at least partially returned to a memorized state, in accordance with an aspect of the present invention;

FIG. 8A is an elevational view of an alternate intervertebral prosthetic device embodiment wherein the arthroplasty implant is stapled to one of two vertebral bodies defining the intervertebral space, in accordance with an aspect of the present invention;

FIG. 8B is an elevational view of an alternate intervertebral prosthetic device embodiment wherein the arthroplasty implant is stapled to the vertebral bodies using two staples, in accordance with an aspect of the present invention;

FIG. 8C is an elevational view of another alternate intervertebral prosthetic device embodiment wherein the arthroplasty implant is affixed to the inferior vertebral body using a single staple design such as illustrated in FIG. 4 and is affixed to the superior vertebral body using a three prong staple design, in accordance with an aspect of the present invention;

FIG. 8D is an elevational view of a further alternate intervertebral prosthetic device embodiment wherein the arthroplasty implant is affixed to the adjacent vertebral bodies using two offset staples, in accordance with an aspect of the present invention;

FIG. 8E is an elevational view of an alternate intervertebral prosthetic device embodiment wherein the arthroplasty implant is affixed to the adjacent vertebral bodies employing four laterally disposed staples, in accordance with an aspect of the present invention;

FIG. 9 is a plan view of an alternate embodiment of an implant-to-vertebra staple of an intervertebral prosthetic device, in accordance with an aspect of the present invention; and

FIG. 10 is a partial lateral cross-sectional view of an alternate embodiment of an intervertebral prosthetic device wherein the arthroplasty implant is affixed to an adjacent vertebral body employing the implant-to-vertebra staple of FIG. 9, in accordance with an aspect of the present invention.

### BEST MODE FOR CARRYING OUT THE INVENTION

Intervertebral prosthetic devices are disclosed which are configured for disposition within an intervertebral space between a first vertebral body and a second vertebral body. In one example, the intervertebral prosthetic device includes at least one staple comprising a first prong and a second prong, and at least one component configured to reside within the intervertebral space between the first vertebral body and the second vertebral body. The at least one component includes at least one vertebral bearing surface configured to engage at least one of the first vertebral body and the second vertebral body and to facilitate repositioning of the at least one component when positioned within the intervertebral space. Further, the at least one component is configured to receive the first prong of the at least one staple. When the at least one component is disposed in the intervertebral space, the at least one staple engagably couples the vertebral bearing surface of the at least one component to the at least one first vertebral body and second vertebral body, with the first prong thereof being received by the at least one component and the second prong engaging the at least one vertebral body and second vertebral body.

In various embodiments described herein, the at least one component is part of a spinal arthroplasty implant comprising a first vertebral support plate and a second vertebral support plate, each of which has at least one prong-receiving opening, hole, receptacle, etc. Further, the intervertebral prosthetic device includes two or more staples, with a first prong of each staple being configured to engage a respective prong-receiving opening in one of the vertebral support plates of the arthroplasty implant. Still more particularly, the two or more staples may be formed of a shape memory material, wherein the first and second prongs of each staple are substantially parallel in a deformed state and are angled toward one another when transformed towards a memorized state. Thus, the shape memory material staples are employed in the deformed state to engagably couple the arthroplasty implant to the first vertebral body and the second vertebral body, and then allowed to transform to their memorized state, thereby applying a compressive force between the vertebral support plate of the arthroplasty implant and the respective first vertebral body and second vertebral body.

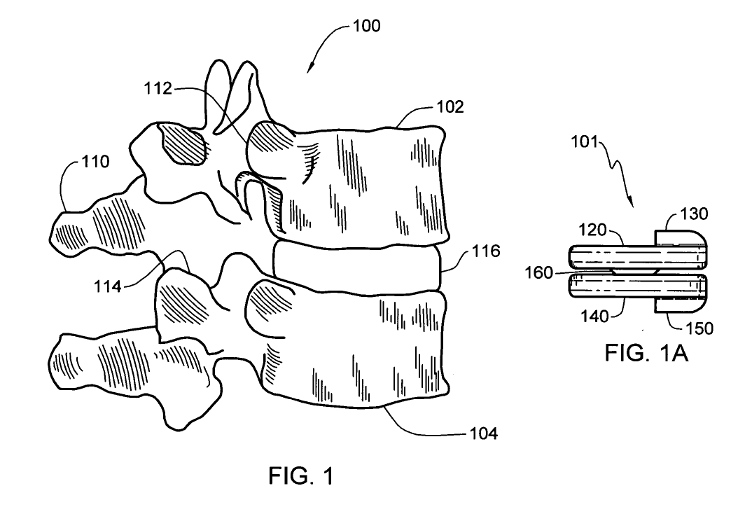

The above-outlined aspects of the present invention, as well as further aspects thereof, are described in greater detail below with reference to FIGS. 1-10.

FIG. 1 illustrates a lateral view of a portion of a spine, generally denoted 100, comprising two vertebrae or vertebral bodies 102, 104, with a disc 116 shown therebetween. Each vertebral body 102, 104 comprises a generally cylindrical body that contributes to the primary weight-bearing portion of spine 100. As shown, each vertebral body 102, 104 further includes various boney processes 110, 112 extending posterior to the body. Adjacent vertebral bodies, 102, 104 move relative to each other via facet joints 114, and due to the flexibility of disc 116.

Each vertebral body 102, 104 comprises an outer cortical rim composed of cortical bone, with an inner cancellous bone disposed within the cortical rim. The cortical rim is often referred to as the apophyseal rim or apophyseal ring. Further, the cancellous bone is softer than the cortical bone of the cortical rim. It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., the structures described above in conjunction with FIG. 1.

If intervertebral disc 116 is diseased, degenerated, damaged, or otherwise in need of replacement, the disc can be at least partially removed and replaced with an intervertebral prosthetic disc such as illustrated in FIG. 1A. The intervertebral disc can be removed via a discectomy, or a similar surgical procedure, well known in the art. Removal of intervertebral disc material results in the formation of an intervertebral space (not shown) between the two adjacent vertebral bodies.

FIG. 1A depicts one embodiment of a spinal arthroplasty device 101 such as disclosed in the above-incorporated, co-pending United States Patent Application entitled, “Intervertebral Spinal Implant Devices and Methods of Use”. Briefly described, spinal arthroplasty device 101 includes three main components, i.e., a first support plate 120, a second support plate 140, and a nucleus 160. Nucleus 160 is positioned between support plates 120, 140 and forms a sliding interface allowing sliding motion of the first and second support plates 120, 140 relative to each other. First support plate 120 is a superior vertebral support plate, while second support plate 140 is an inferior vertebral support plate. Each support plate 120, 140 includes a respective anchor or keel 130, 150 that fits within a corresponding recess (not shown) formed in the cancellous bone of vertebral bodies 102, 104. Employing this device, vertebral bodies 102, 104 typically require a certain amount of surgical preparation to accept the device, and this may include contouring to match the bone interface surfaces and/or bone removal to create recesses into which anchors or keels 130, 150 are to be inserted.

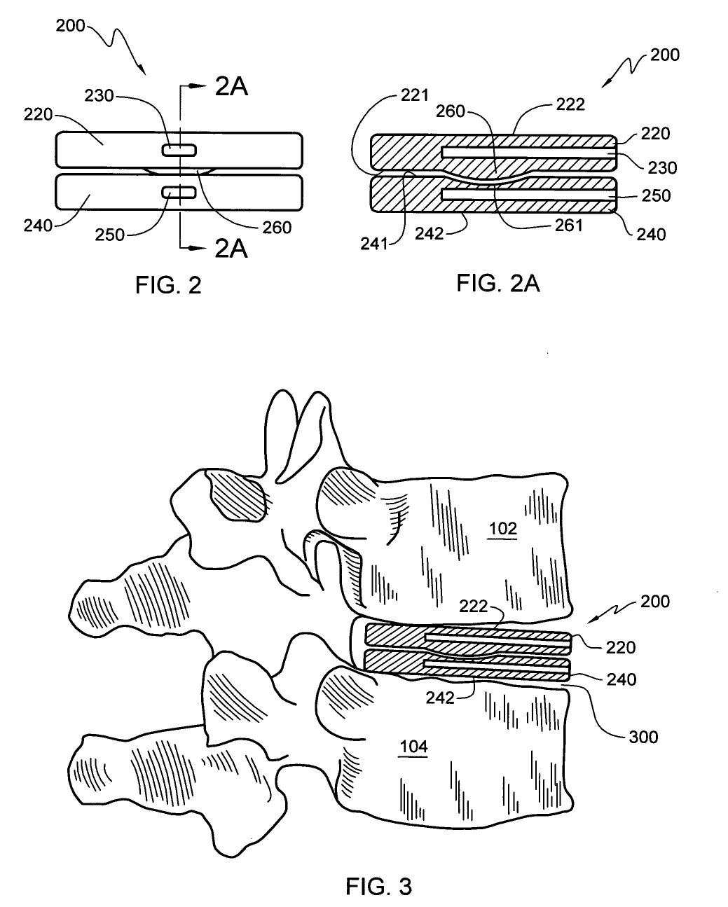

FIGS. 2 & 2A depict one embodiment of an arthroplasty implant 200 of an intervertebral prosthetic device, in accordance with an aspect of the present invention. By way of example only, implant 200 is shown as a multi-component implant, including a first vertebral support plate 220, and a second vertebral support plate 240, with a nucleus 260 formed integral with the first vertebral support plate 220. Nucleus 260 is a protrusion formed in an articulation surface 221 of first vertebral support plate 220, and a correspondingly configured depression 261 is formed in an articulation surface 241 of second vertebral support plate 240. Together, the interfacing protrusion 260 and depression 261 of the first and second vertebral support plates 220, 240 form an articulating joint. The arthroplasty implant, with the articulating joint, is sized and configured for deposition within an intervertebral space between adjacent vertebral bodies 102, 104 as shown in FIG. 3.

The articulating joint provides relative pivotal and rotational movement between the adjacent vertebral bodies to maintain or restore motion substantially similar to the normal bio-mechanical motion provided by a natural intervertebral disc. Specifically, the articulating vertebral support plates 220, 240 are permitted to pivot relative to one another about a number of axes, including a lateral or side-to-side pivotal movement about a longitudinal axis, and an anterior-posterior pivotal movement about a transverse axis. Further, it should be understood that these articular components are permitted to pivot relative to one another about any axis that lies in a plane that intersects the longitudinal axis and the transverse axis. Additionally, the articular components are preferably permitted to rotate relative to one another about a rotational axis. Although the articulating joint is illustrated and described as providing a specific combination of articulating motion, it should be understood that other combinations of articulating movement are also possible and are contemplated as falling within the scope of the present invention. Further, it should be understood that other types of arthroplasty implants allowing articulating movement are also contemplated, including for example, single-component and three-component or more prosthetic discs.

Although the articulating vertebral support plates 220, 240 of the prosthetic joint may be formed from a wide variety of materials, including metal-containing materials, polymer materials, or composite materials that include metals, polymers, or combinations of metals and polymers, in one embodiment, the components are formed of a cobalt-chrome-molybdenum metallic alloy (ASTM F-999 or F-75). However, in alternative embodiments, the support plates may be formed of other materials, including ceramic material, other metallic material, such as titanium or stainless steel, polymeric material (such as polyurethane material, polyolefin material, polyether material, silicone material, or a combination thereof), or any other biocompatible material that would be apparent to one of ordinary skill in the art. Further, depending upon the implant configuration, nucleus 260 may comprise the same material as vertebral support plates 220, 240, or a different material. For example, nucleus 260 could be an implantable grade PEEK material. One example of a suitable medical grade material is marketed as PEEK® Optima available from Invibio, Inc., of Greenville, S.C., USA.

As shown in FIGS. 2, 2A & 3, vertebral support plates 220, 240 each include a vertebral bearing surface 222, 242, which is shown to be free of any keel, anchor, spike, etc. designed to securely affix the implant relative to the adjacent vertebrae. Advantageously, this facilitates positioning of arthroplasty implant 200 within the intervertebral space of a patient and then the ready adjusting of the implant within the space as desired. In addition to the clear placement advantages, the keel-less or anchor-less vertebral bearing surfaces 222, 242 are beneficial with surgical techniques requiring an inter-operative flexion/extension radiograph, or other simulation of the implant's range of motion in situ. The implant position could be readjusted until the arthroplasty implant is located within the anatomic center of rotation for the surrounding anatomy.

Vertebral contact surfaces 222, 242 are designed to be in direct physical contact with the respective vertebral bodies and may be coated or textured to promote osteointegration. For example, a bone-growth promoting substance such as, for example, hydroxyapatite coating formed of calcium phosphate may be employed. Additionally, the vertebral contacting surfaces 222, 242, may be roughened prior to being coated with the bone-growth promoting substance to further enhance bone on-growth. Such surface roughening may be accomplished by way of, for example, acid etching, knurling, application of a bead coating (e.g., cobalt chrome beads), application of a roughening spray (e.g., titanium plasma spray (TPS)), laser blasting, or other methods of roughening that are known to one skilled in the art.

Also shown in FIGS. 2, 2A & 3 are rectangular-shaped openings 230, 250 extending from the anterior of arthroplasty implant 200 into the implant a distance at least equal to the center of the implant. These openings 230, 250 are configured to respectively receive a prong of two different staples employed to affix the arthroplasty implant relative to the vertebral bodies 102, 104 once positioned within the intervertebral space. Openings 230, 250 may be machined as holes in the perimeter surface of the respective end plates, and may be placed either anterior, lateral or oblique. As explained further below, by extending openings 230, 250 into at least the center region of the arthroplasty implant (i.e., within a region over nucleus 260 or depression 261), compressive loading resulting from the use of shape memory staples, in one embodiment, is more evenly applied across the respective vertebral contact surfaces 222, 242 between the vertebral support plates 220, 240 and the vertebral bodies 102, 104.

As shown in FIG. 3, without the use of the implant-to-vertebra affixation staples, described further below, gaps 300 may exist between the vertebral contact surfaces 222, 242 and the respective vertebral bodies 102, 104.

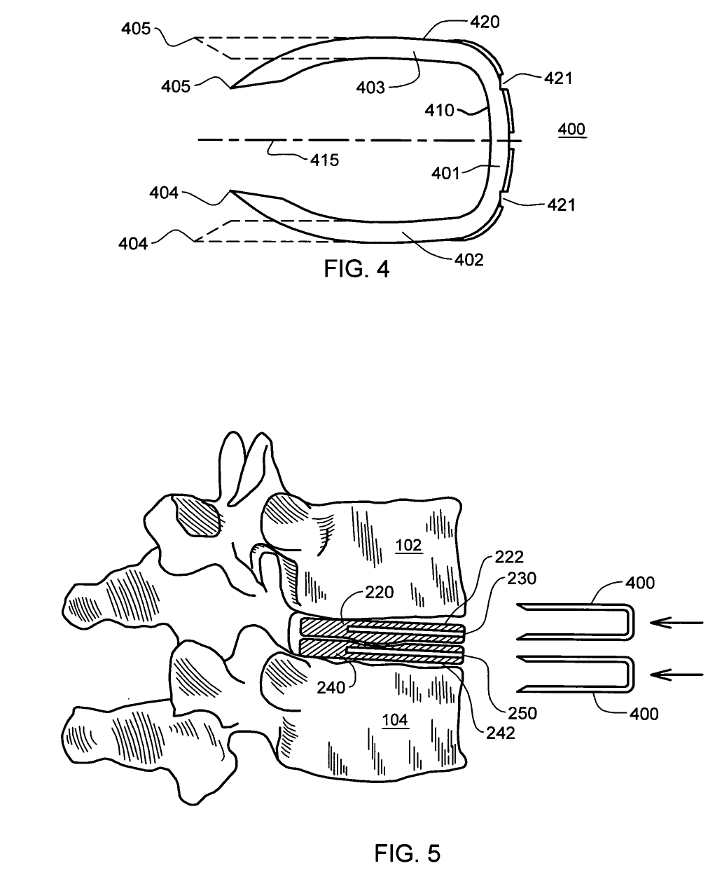

Thus, in accordance with an aspect of the present invention, the intervertebral prosthetic device further includes at least one staple having at least two prongs, that is, a first prong and a second prong. Each staple may be manufactured from a conventional implant material, such as stainless steel or titanium, or in another embodiment, the staples may be manufactured of shape memory material or alloy, such as nickel titanium, to provide a compressive fixation which does not interfere with the implant's articulation surfaces. One example of a shape memory alloy is Nitinol sold by Memry Corporation of Melno Park, Calif. One embodiment of a shape memory staple, generally denoted 400, is illustrated in FIG. 4.

Staple 400 is generally U-shaped with a crossbar 401 connecting prongs 402 and 403. In this embodiment, prong 202 has a pointed tip 204, while prong 203 has a pointed tip 205. Depending on the orientation of staple 400, pointed tips 204, 205 may facilitate insertion of the respective prong into the cortical rim of one of the vertebral bodies. It should be understood, however, that tips 404, 405 may have a variety of configurations. Staple 400, which is symmetrical about a center line 415, includes an inner surface 410 and an outer surface 420. In the illustrated embodiment, crossbar 401 includes notches 421 on outer surface 420 for facilitating final seating of the staple, for example, into bone. Notches 421 allow a surgeon to drive each prong independently as necessary. Although two prongs are shown, those skilled in the art will understand from the following description, that a staple with three, four or more prongs could alternately be employed. Various dimensions and tolerances for the staples to be employed in the intervertebral prosthetic device described herein will be apparent to a person of ordinary skill in the art. While various examples of staple configurations are described further below, certain general features are reviewed here.

As noted, the staples may be a shape memory material, for example, made of Nitinol, which is a biocompatible, shape memory metal alloy of titanium and nickel. Such staples are capable of being bent when cooled and transformed to their original shape when reheated. It is also possible to take advantage of the shape memory alloy's ability to transform from its austentic state to a stress-induced martensitic state. The metal changes shape with temperature or under the influence of stress because of crystalline phase changes. Thus, a staple made of a shape memory alloy can be inserted in two different ways as desired. In one embodiment, the shape memory alloy staple is cooled and then deformed while at a temperature less than the transformation temperature at which it is in the martensitic phase. The staple is then inserted in its deformed shape (shown in phantom in FIG. 4) and when heated will reform to its original shape. In the second embodiment, the staple is deformed and inserted while held in the deformed state. In the second embodiment, the shape memory alloy is selected to have a temperature transformation range such that the staple undergoes a transition from austenite to stress-induced martensite under the influence of the deformation forces. Thus, when the staple of the second embodiment is inserted and released it is already at a temperature such that it automatically attempts to reform to its original shape.

The metal's properties at the higher temperature (austenite phase) are similar to those of titanium. The temperature at which the staples will undergo the shape transformation can be controlled by the manufacturing process and the selection of the appropriate alloy composition. Injury to the surrounding tissues should be negligible if the transformation temperature is near body temperature. There is no threat of thermal injury to the spinal cord or nerves, or adjacent vascular structures. Nitinol has a very low corrosion rate and has been used in a variety of medical implants (i.e., orthodontic appliances, stents). Implant studies in animals have shown minimal elevations of nickel in the tissues in contact with the metal, and the levels of titanium are comparable to the lowest levels found in tissues near titanium hip prostheses.

A method of fixing the intervertebral prosthetic device in an intervertebral space, in accordance with aspects of the present invention, is illustrated in the lateral views of FIGS. 5-7. As one example, in FIG. 5 the diseased or degenerated natural intervertebral disc (not shown) has been removed via a discectomy or similar surgical procedure, and a fully assembled arthroplasty implant 200, such as described above in connection with FIGS. 2-3, has been implanted in the intervertebral space via, for example, an anterior approach. Arthroplasty implant 200 again includes a first vertebral support plate 220 and a second vertebral support plate 240, each of which includes a prong-receiving opening 230, 250 a sufficient length to receive a respective prong of the staples 400 to be employed in affixing the arthroplasty implant to the adjacent vertebral bodies. After placement within the intervertebral space, the arthroplasty implant 200 can be re-positioned within the intervertebral space as needed, with the vertebral bearing surfaces 222, 242 roughly in physical contact with the respective vertebral bodies 102, 104, but with gaps 300 present. Advantageously, prong-receiving openings 230, 250 could also be employed as implant inserter engagement holes during insertion of the arthroplasty implant into the intervertebral space. Appropriate dowels, pins, etc., extending from an implant inserter (not shown) could be used to facilitate insertion of the arthroplasty implant.

A number of general surgical instruments are used in the procedure, along with specific implants and instruments for affixing the staples between the implant and the vertebral bodies. The instruments used in this procedure may include one or more of: an implant inserter, a staple awl, a staple opener, a straight staple inserter, an angled staple inserter, a staple impactor, and a staple extractor.

To insert the staples, pilot holes may be made using the staple awl. The pilot holes are made anterior to the mid-body of the vertebrae. The staple awl is inserted part way and position is checked using, e.g., an x-ray or image intensifier. Prior to removal of the staple awl from the pilot holes, an electric cauterizer (Bovie) can be placed in contact with the endcap of the staple awl to minimize bleeding from the pilot holes. In one embodiment, two sets of pilot holes are made, one at each level to accommodate two staples per disc space. The two staples are then inserted, using for example, the straight staple inserter or the angled staple inserter, each with one prong thereof in one of the pilot holes previously made by the staple awl, and another prong in a respective prong-receiving opening 230, 250 formed in the vertebral support plates 220, 240 of the arthroplasty implant 200. The inserter may be tapped with a mallet to facilitate placement of the staple in the respective vertebral body. The staple is then released from the inserter and the instrument is removed. If further seating of the staple is required, the staple impactor may be used in conjunction with a mallet for final seating of a staple prong into either the bone, or the arthroplasty implant. The aforementioned steps are repeated for each staple.

As noted, in one embodiment, staple 400 is a shape memory alloy staple which has a deformed state when cooled, shown in phantom in FIG. 4, and a memorized state when heated past a transformation temperature. As shown in FIG. 4, in the memorized state the prongs of the staple are closer together than in the deformed state. In the deformed state, the prongs are substantially parallel to facilitate insertion of the staple into the vertebral body and the arthroplasty implant.

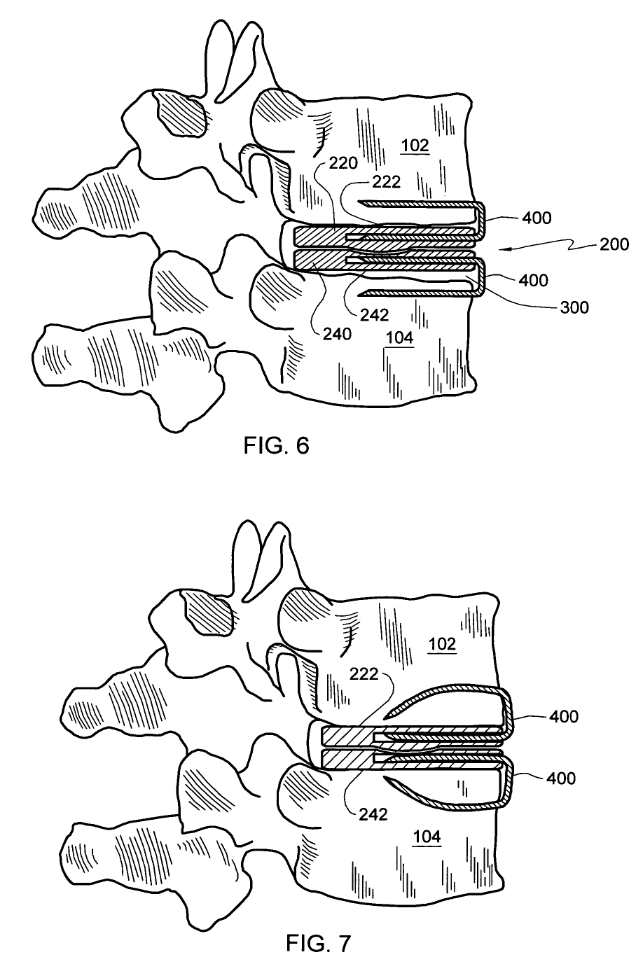

FIG. 6 illustrates a lateral view of the spinal structure of FIG. 5, soon after the two shape memory staples have been inserted into a respective vertebral body and a respective vertebral support plate of the arthroplasty implant. In this view, gaps 300 are still present. As noted above, the transformation temperature is selected to be below the body temperature of the patient so that the prongs will attempt return to their memorized state, as shown in FIG. 7. By returning the prongs to the memorized state, a compressive force is applied by the prongs of each staple between a respective vertebral support plate and the adjacent vertebral body, thereby forcing each vertebral contact surface 222, 242 of the arthroplasty implant 200 into good physical contact with the respective endplate surface of the adjacent vertebral body. The vertebral support plates (i.e., articulation components) are further secured to the upper and lower vertebrae via bone growth onto the vertebral bearing surfaces of the components that are in direct contact with the vertebral bone, which is facilitated by the compressive force applied by the shape memory staples. The bone on-growth provides further resistance to the migration or displacement of the prosthetic joint and prevents expulsion of the prosthetic joint from the intervertebral space.

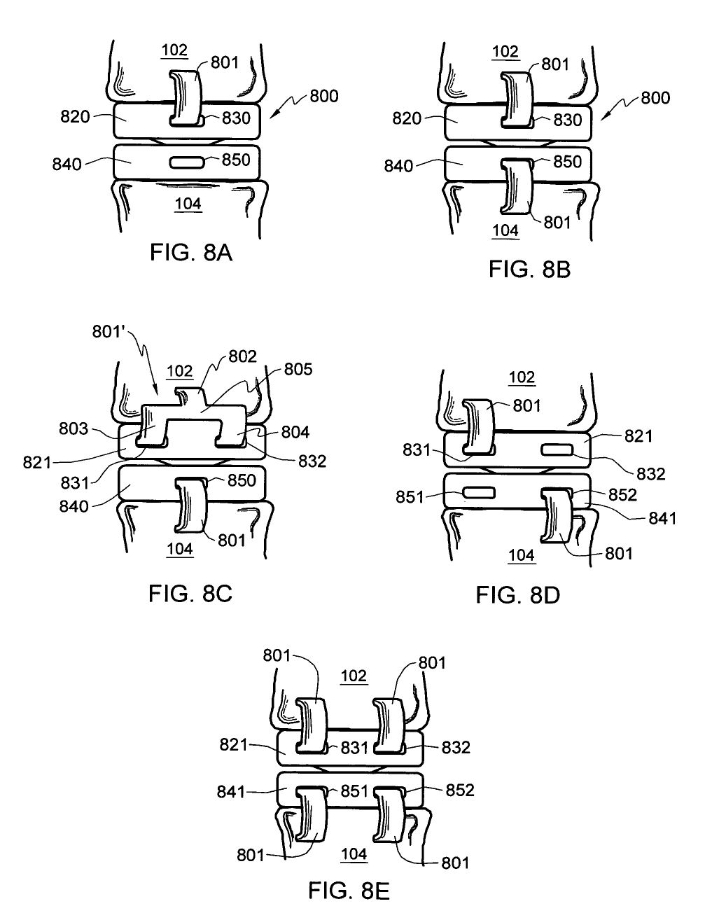

FIGS. 8A-8E are anterior views of alternate embodiments of an intervertebral prosthetic device, in accordance with aspects of the present invention. These figures illustrate various approaches to affixing the arthroplasty implant to the superior and inferior vertebral bodies 102, 104. Surgical procedures as outline above in connection with FIGS. 5-7 could be employed, with one difference between the embodiments being the number and placement of the pilot holes in the adjacent vertebral bodies. As noted above, each staple may either be a conventional staple of biocompatible material, or a staple formed of a shape memory material. Depending upon the number of staples to be employed, the vertebral support plates of the arthroplasty implant are preconfigured with one or more prong-receiving openings, preferably extending at least mid-way into the arthroplasty implant.

In FIG. 8A, a single staple 801 is employed to affix the arthroplasty implant 800 to vertebral body 102. In this embodiment, first vertebral support plate 820 includes a prong-receiving opening 830, while second vertebral support plate 840 includes a prong-receiving opening 850. In FIG. 8B, two staples 801 are employed to affix the vertebral support plates 820, 840 of the arthroplasty implant 800 to the respective vertebral bodies 102, 104, in a manner described above in connection with FIGS. 5-7. In particular, a first staple 801 includes a first prong residing within prong-receiving opening 830 in vertebral support plate 820 and a second prong impacted into vertebral body 102, while a second staple 801 has a first prong disposed within prong-receiving opening 850 of vertebral support plate 840 and a second prong impacted into vertebral body 104. FIG. 8C depicts an alternate embodiment wherein a three-prong staple 801′ is illustrated with a first prong 802 extending into the vertebral body, and a second and third prong 803, 804 branching from the crossbar 805 and extending into a superior vertebral support plate 821 of the arthroplasty implant. In this embodiment, a single two-prong staple 801 is employed to affix the inferior vertebral support plate 840 of the arthroplasty implant to the inferior vertebral body 104. To accommodate second and third prongs 803, 804, a first lateral prong-receiving opening 831 and a second lateral prong-receiving opening 832 are defined in the first vertebral support structure 821.

A similar disposition of prong-receiving openings is employed in both the first vertebral support plate 821 and second vertebral support plate 841 in the embodiment of FIG. 8D. In this embodiment, vertebral support plate 821 includes a first prong-receiving opening 831 and a second prong-receiving opening 832, while second vertebral support plate 841 includes a first prong-receiving opening 851 and a second prong-receiving opening 852. Further, this embodiment employs two two-prong staples at opposite sides of the arthroplasty implant affixed to the symmetrically offset prong-receiving openings of the implant to couple the implant to the respective vertebral bodies 102, 104. In FIG. 8E, four two-prong staples 801 are used, with each two-prong staple 801 engaging a respective prong-receiving opening 831, 382, 851, 852 in the vertebral support plates 821, 841 of the arthroplasty implant, as well as one of the adjacent vertebral bodies 102, 104. As a variation on the embodiment of FIG. 8E, two four-prong staples (not shown) could be employed, with a crossbar connecting two pairs of prongs in a manner described in various ones of the initially-incorporated U.S. patents and applications.

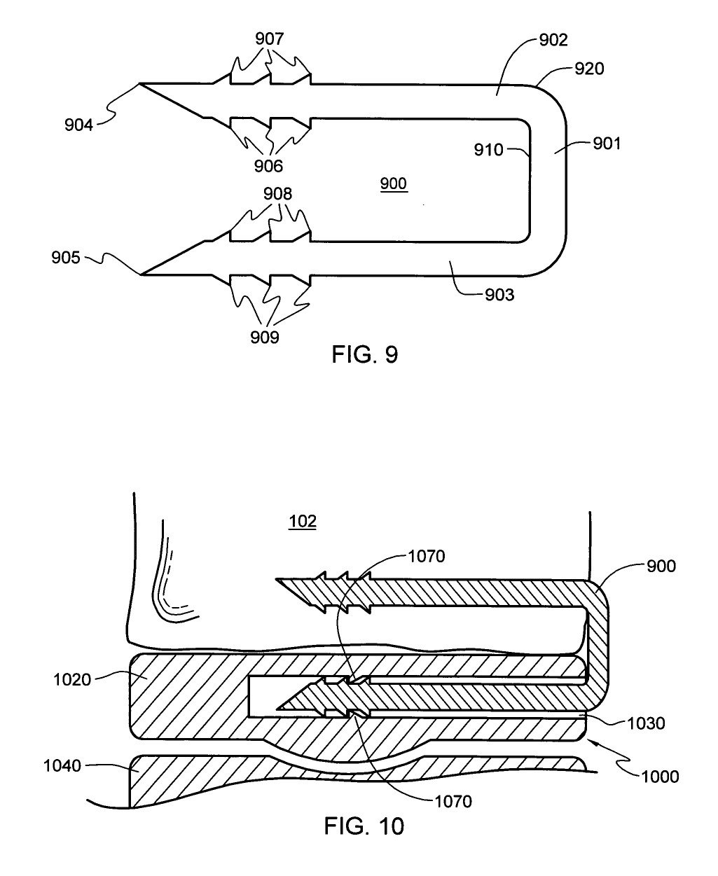

FIG. 9 depicts still another variation for the intervertebral prosthetic device described herein. In this variation, a staple 900 is illustrated which is generally a U-shaped staple having a crossbar 901 connecting a first prong 902 and a second prong 903. Staple 900 may comprise a conventional material, or be formed of a shape memory material such as described above in connection with FIG. 4. Staple 900 has an inner surface 910 and an outer surface 920. Staple prong 902 has a pointed tip 904, while staple prong 903 has a pointed tip 905. Again, it should be understood that tips 904, 905 may have a variety of configurations, and may be the same or different. Prong 902 has barbs 906 on inner surface 910 and barbs 907 on outer surface 920. Similarly, prong 903 has barbs 908 on inner surface 910, and barbs 909 on outer surface 920. The number, size and shape of barbs 906, 907, 908 & 909 can vary between the inner and outer surfaces 910, 920, as well as between the prongs 902, 903. For example, prong 902 may include barbs 906, 907, while prong 903 may be configured without barbs, such as prongs 402 & 403 of the staple embodiment illustrated in FIG. 4. Barbs 906, 907, 908 & 909 may aid in the prevention of staple back-out. Further, having barbs on both the inner surface 910 and outer surface 920 of each prong 902, 903 allows the use of shorter barbs in the direction transverse to the longitudinal axis of each prong.

FIG. 10 is a partial cross-sectional lateral view of an arthroplasty implant 1000 shown disposed in an intervertebral space defined between adjacent vertebral bodies, with only vertebral body 102 being shown. In this embodiment, the intervertebral prosthetic device 1000 includes a first vertebral support structure 1020 having a prong-receiving opening 1030 defined therein extending at least half-way into the vertebral support structure. Further, in the cross-sectional view illustrated, a multi-barbed, two-pronged staple 900, such as described above in connection with FIG. 9, is employed. The arthroplasty implant 1000 is configured with a protrusion 1070 on an inner surface thereof defining the prong-receiving opening 1030. Protrusion 1070 is angled in the prong insertion direction, and forms a shoulder which engages the barbs on the inner and outer surfaces of staple 900 should the staple attempt to back-out. As noted, staple 900 may be formed of a conventional material, or may be formed of a shape memory material to facilitate compressive forcing of the arthroplasty implant into good physical contact with the adjacent vertebral bodies defining the intervertebral space. In each embodiment, however, the vertebral support plates include vertebral bearing surfaces which are configured to allow repositioning of the arthroplasty implant within the intervertebral space prior to engagable coupling of the first and second vertebral bodies employing the one or more staples.

Advantageously, the intervertebral prosthetic device and method of disc replacement described above allow inter-operative flexion/extension radiography, or other simulation of the implant's range of motion in situ prior to final positioning of the device. The implant may be placed into the intervertebral space fully assembled and then repositioned within the space until the device is located within the anatomic center of rotation for the surrounding anatomy. Further, because the vertebral bearing surfaces of the implant are keel-less or anchor-less, the device could be employed with ceramic support plates. Once the device is in proper position, staples, such as shape memory staples, can be inserted into the blind openings in the vertebral support plates and into the adjacent vertebral bodies. Shape memory staples have the further advantage of providing a compressive force between the vertebral support plates of the implant and the vertebral endplates of the surrounding vertebrae.

Although preferred embodiments are depicted and described in detail herein, it will be apparent to those skilled in the relevant art that various modifications, additions, substitutions and the like can be made without departing from the spirit of the invention and these are therefore considered to be within the scope of the invention as defined in the following claims. For example, various components in the embodiments described herein are referred to as “superior” and “inferior” for illustrative purposes only, in that one or more of the features described as part of or attached to a respective half may be provided as part of or attached to the other half in addition to or in the alternative.

What is claimed is:

1. An intervertebral prosthetic device comprising:
at least one staple comprising at least two prongs including a first prong and a second prong;
at least one component configured to reside within an intervertebral space between a first vertebral body and a second vertebral body, the at least one component including at least one vertebral bearing surface configured to engage at least one of the first vertebral body and the second vertebral body and to facilitate repositioning of the at least one component within the intervertebral space, and wherein the at least one component is configured to receive the first prong of the at least one staple; and
wherein when the at least one component is disposed in the intervertebral space, the at least one staple couples the at least one vertebral bearing surface of the at least one component to the at least one first vertebral body and second vertebral body, with the first prong thereof being received by the at least one component and the second prong engaging the at least one first vertebral body and second vertebral body.

2. The intervertebral prosthetic device of claim 1, wherein the at least one staple is formed of a shape memory material.

3. The intervertebral prosthetic device of claim 2, wherein the at least one staple is formed of a shape memory alloy having a memorized state and a deformed state, wherein the first and second prongs are disposed closer to one another in the memorized state than in the deformed state.

4. The intervertebral prosthetic device of claim 3, wherein the at least one staple couples the at least one component to the at least one first vertebral body and second vertebral body when in the deformed state and thereafter transforms to the memorized state, wherein the transitioning of first prong and the second prong to the memorized state results in compressive force being applied between the at least one component and the at least one first vertebral body and second vertebral body.

5. The intervertebral prosthetic device of claim 4, wherein the first and second prongs are substantially parallel in the deformed state, and are angled toward one another when transformed towards the memorized state.

6. The intervertebral prosthetic device of claim 2, wherein the second prong includes a distal portion defining a pointed tip to aid insertion of the second prong into the at least one first vertebral body and second vertebral body.

7. The intervertebral prosthetic device of claim 1, wherein the first prong is configured differently than the second prong.

8. The intervertebral prosthetic device of claim 1, wherein each staple of the at least one staple comprises a first prong and a second prong interconnected by a crossbar, the crossbar including a plurality of notches, the plurality of notches permitting independent seating of the first and second prongs.

9. The intervertebral prosthetic device of claim 1, wherein at least one of the first prong and the second prong includes a plurality of barbs extending therefrom.

10. The intervertebral prosthetic device of claim 9, wherein the first prong includes barbs extending therefrom, and wherein the at least one component is configured with at least one opening sized to receive the first prong and extending into the at least one component to at least a center region thereof, and wherein the at least one component includes at least one protrusion on an inner wall thereof defining the at least one opening, the at least one protrusion being configured to permit insertion of the first prong into the at least one opening, and inhibit removal of the at least one prong therefrom, wherein the at least one protrusion is positioned to engage one or more of the barbs of the first prong.

11. The intervertebral prosthetic device of claim 1, wherein the at least one staple further comprises a third prong disposed adjacent to the first prong to define a pair of prongs, wherein the pair of prongs and the second prong are interconnected by a crossbar.

12. The intervertebral prosthetic device of claim 1, wherein the at least one component comprises a first component and a second component, the first component including a first vertebral bearing surface configured to engage the first vertebral body and the second component including a second vertebral bearing surface configured to engage the second vertebral body, and wherein the at least one staple comprises at least two staples, each staple including a first prong and a second prong, wherein a first staple couples the first component to the first vertebral body and a second staple couples a second component to the second vertebral body.

13. The intervertebral prosthetic device of claim 12, wherein the first component comprises a first vertebral support plate and the second component comprises a second vertebral support plate, wherein the first vertebral support plate is translatable relative to the second vertebral support plate.

14. The intervertebral prosthetic device of claim 13, wherein the at least one component is an arthroplasty implant, the arthroplasty implant comprising the first vertebral support plate and the second vertebral support plate.

15. The intervertebral prosthetic device of claim 14, wherein the first vertebral bearing surface and the second vertebral bearing surface are each configured to facilitate repositioning of the arthroplasty implant within the intervertebral space prior to coupling thereof to the first and second vertebral bodies employing the first and second staples.

16. The intervertebral prosthetic device of claim 12, wherein the first component and the second component are each configured with at least one opening sized to receive the first prong of the respective first and second staples.

17. The intervertebral prosthetic device of claim 16, wherein the at least one opening is positioned to intersect a center axis of the at least one component.

18. The intervertebral prosthetic device of claim 16, wherein each opening sized to receive the first prong of the respective first and second staples extends into the respective first component and second component to at least a center region thereof, and wherein the first and second staples are formed of a shape memory material and apply a compressive force between the first component and the first vertebral body and a compressive force between the second component and the second vertebral body when positioned within the first and second components and the first and second vertebral bodies, with the intervertebral prosthetic device disposed within the intervertebral space.

19. The intervertebral prosthetic device of claim 12, wherein at least one of the first component and the second component includes multiple prong-receiving lateral openings, each sized to receive a staple prong.

20. The intervertebral prosthetic device of claim 19, wherein the first component and the second component each include multiple prong-receiving lateral openings, each sized to receive a staple prong, and wherein the respective first and second staples engage prong-receiving lateral openings on opposite sides of the first and second components.

21. The intervertebral prosthetic device of claim 19, wherein the at least one staple comprises at least four staples, each staple including a first prong and a second prong, and wherein a first staple and a third staple couple the first component to the first vertebral body, and a second staple and a fourth staple couple the second component to the second vertebral body.

22. The intervertebral prosthetic device of claim 1, wherein the at least one component is an arthroplasty implant, the arthroplasty implant comprising a first component and a second component, wherein the first component includes a first vertebral bearing surface, adapted to engage the first vertebral body, and includes a first articular surface, and the second component includes a second vertebral bearing surface, adapted to engage the second vertebral body, and includes a second articular surface, the first and second articular surfaces cooperating to permit articulating motion between the first and second components.

23. The intervertebral prosthetic device of claim 22, wherein the first articular surface includes a projection, and the second articular surface includes a recess, and wherein at least a portion of the projection is disposed within the recess to permit articulating motion between the first and second components.

24. An intervertebral prosthetic device comprising:
at least one staple comprising at least two prongs, including a first prong and a second prong, the at least one staple being formed of a shape memory material having a memorized state and a deformed state;
an arthroplasty implant configured to reside within an intervertebral space between a first vertebral body and a second vertebral body, the arthroplasty implant including at least one vertebral support plate configured for engaging at least one of the first vertebral body and the second vertebral body, the at least one vertebral support plate being configured to receive the first prong of the at least one staple; and
wherein when the arthroplasty implant is disposed in the intervertebral space, the at least one staple couples the at least one vertebral support plate of the arthroplasty implant to the at least one first vertebral body and second vertebral body when in the deformed state, and thereafter transitions to the memorized state resulting in compressive force being applied between the at least one vertebral support plate and the at least one first vertebral body and second vertebral body.

25. The intervertebral prosthetic device of claim 24, wherein the at least one vertebral support plate comprises a first vertebral support plate and a second vertebral support plate, and wherein the at least one staple comprises a first staple and a second staple, wherein the first staple couples the first vertebral support plate to the first vertebral body and the second staple couples the second vertebral support plate to the second vertebral body, and wherein when the arthroplasty implant is disposed in the intervertebral space, with the first and second staples coupling the first and second vertebral support plates to the first and second vertebral bodies, and the first and second staples in the memorized state, compressive force is applied by the first staple between the first vertebral support plate and the first vertebral body, and by the second staple between the second vertebral support plate and the second vertebral body.

26. The intervertebral prosthetic device of claim 25, wherein the first vertebral support plate and the second vertebral support plate each include at least one opening sized to receive the first prong of the respective first and second staples, the at least one opening in each vertebral support plate extending at least to a center region thereof.

27. The intervertebral prosthetic device of claim 25, wherein the first prong and the second prong of each staple are interconnected by a crossbar, the crossbar including a plurality of notches, the plurality of notches permitting independent seating of the first and second prongs.

28. The intervertebral prosthetic device of claim 25, wherein at least one of the first prong and the second prong of the first and second staples includes a plurality of barbs extending therefrom.

29. The intervertebral prosthetic device of claim 28, wherein the first prong of the first and second staples includes barbs extending therefrom, and wherein the first vertebral support plate and second vertebral support plate are each configured with at least one prong-receiving opening sized to receive the first prong of the respective staple and extending into a center region thereof, and wherein each includes at least one protrusion on an inner wall thereof defining the at least one prong-receiving opening, the at least one protrusion being configured to permit insertion of the first prong into the at least one opening, and inhibit removal of the first prong therefrom, and wherein the at least one protrusion is sized and configured to engage one or more of the barbs of the first prong.

30. The intervertebral prosthetic device of claim 25, wherein at least one staple of the first staple and the second staple comprises a third prong disposed adjacent to one of the first prong and the second prong to define a pair of prongs, wherein all prongs of the at least one staple having the third prong are interconnected by a crossbar.

31. The intervertebral prosthetic device of claim 25, wherein the first vertebral support plate and the second vertebral support plate are translatable relative to each other.

32. The intervertebral prosthetic device of claim 31, wherein the first vertebral support plate includes a first vertebral bearing surface and the second vertebral support plate includes a second vertebral bearing surface, wherein the first vertebral bearing surface and the second vertebral bearing surface are configured to facilitate repositioning of the arthroplasty implant within the intervertebral space prior to coupling thereof to the first and second vertebral bodies employing the first and second staples.

33. The intervertebral prosthetic device of claim 32, wherein the first vertebral bearing surface and the second vertebral bearing surface are further configured to facilitate osteointegration with the first vertebral body and the second vertebral body, respectively, when the arthroplasty implant is disposed within the intervertebral space with the first and second staples coupling the arthroplasty implant to the first and second vertebral bodies.

34. The intervertebral prosthetic device of claim 32, wherein the first vertebral support plate further comprises a first articular surface and the second vertebral support plate further comprises a second articular surface, the first and second articular surfaces cooperating to permit articulating motion between the first and second vertebral support plates.

35. The intervertebral prosthetic device of claim 34, wherein the first articular surface includes a projection, and the second articular surface includes a recess, and wherein at least a portion of the projection is disposed within the recess to permit articulating motion between the first and second vertebral support plates.

36. A method of providing a prosthetic intervertebral disc replacement, the method comprising:
obtaining an intervertebral prosthetic device comprising at least one staple and an arthroplasty implant, the at least one staple including a first prong and a second prong, and being formed of a shape memory material having a memorized state and a deformed state, and the arthroplasty implant being configured to reside within an intervertebral space between a first vertebral body and a second vertebral body, the arthroplasty implant including at least one vertebral support plate configured for engaging at least one of the first vertebral body and the second vertebral body, the at least one vertebral support plate being configured to receive the first prong of the at least one staple;
preparing the intervertebral disc space to receive the intervertebral prosthetic device;
positioning the arthroplasty implant within the intervertebral space;
inserting the at least one staple in the deformed state into engagable contact with the at least one vertebral support plate and the at least one first vertebral body and second vertebral body; and
allowing the at least one staple to at least partially return to the memorized state, thereby applying a compressive force between the at least one vertebral support plate and the at least one first vertebral body and second vertebral body.

37. The method of claim 36, further comprising placing the at least one staple in the deformed state prior to inserting thereof into engagable contact with the at least one vertebral support plate and the at least one first vertebral body and second vertebral body.

38. The method of claim 36, wherein the obtaining further comprises obtaining the intervertebral prosthetic device with the at least one vertebral support plate comprising at least one vertebral bearing surface configured to engage the at least one of the first vertebral body and the second vertebral body, and to facilitate repositioning of the arthroplasty implant within the intervertebral space prior to the inserting of the at least one staple.

39. The method of claim 36, wherein the at least one staple further includes a crossbar interconnecting the at least one prong and the second prong, the crossbar including a plurality of notches, and wherein the method further comprises independently seating at least one of the first prong and the second prong by engaging at least one of the plurality of notches in the crossbar of the at least one staple.
